(19) United States
(12) Patent Application Publication
(10) Pub. No.: US 2026/0182951 A1
(43) Pub. Date: Jul. 2, 2026

(54) IMAGE PROCESSING APPARATUS, MEDICAL IMAGE ACQUISITION APPARATUS, AND PROGRAM

(71) Applicant: FUJIFILM Corporation, Tokyo (JP)

(72) Inventor: Yukiya MIYACHI, Kanagawa (JP)

(73) Assignee: FUJIFILM Corporation, Tokyo (JP)

(21) Appl. No.: 19/547,856

(22) Filed: Feb. 24, 2026

Related U.S. Application Data
(63) Continuation of application No. PCT/JP2024/034231, filed on Sep. 25, 2024.

(30) Foreign Application Priority Data
Sep. 28, 2023 (JP) 2023-168674

(51) Int. Cl. A61B 8/08; A61B 8/00; G06T 5/50

(52) U.S. Cl. CPC A61B 8/0825; A61B 8/403; A61B 8/463; G06T 5/50; G06T 2207/10132; G06T 2207/20212; G06T 2207/30068

(57) ABSTRACT

An image processing apparatus including a processor configured to: create a steered transmission/reception image of a breast obtained by an ultrasound beam transmitted and received, via a compression plate, by beam steering with a predetermined steering angle using an ultrasound probe and a non-steered transmission/reception image of the breast obtained by the ultrasound beam transmitted and received, via the compression plate, by non-beam steering using the ultrasound probe, in a state where the breast is compressed; and create a compound image using the steered and non-steered transmission/reception images. The steered and non-steered transmission/reception images are created by using, as a main frequency component, a frequency component in a range in which an amplitude of an artifact caused by a sidelobe of the ultrasound beam is half or less of an amplitude of the ultrasound beam transmitted to create the steered transmission/reception image.

### CROSS REFERENCE TO RELATED APPLICATIONS

This application is a continuation of International Application No. PCT/JP2024/034231, filed on Sep. 25, 2024, which claims priority from Japanese Patent Application No. 2023-168674, filed on Sep. 28, 2023. The entire disclosure of each of the above applications is incorporated herein by reference.

### BACKGROUND

### Technical Field

The present disclosure relates to an image processing apparatus, a medical image acquisition apparatus, and a program.

### Related Art

JP2005-125080A discloses a method for observing an abnormal part in different types of images.

The method includes a stage of scanning an object using a first imaging system to obtain at least a first image of the object, a stage of determining coordinates of a region of interest (ROI) that is observable on the first image, in which the ROI includes an abnormal part, and a stage of scanning the object using a second imaging system using the coordinates of the ROI.

JP2008-514264A discloses a method of executing ultrasound image diagnosis of a breast.

In the method, the breast is received between a first compression plate and a second compression plate to compress the breast. The breast spreads from a chest wall on a proximal end side of a patient to a nipple on a distal end side. A portion of the breast close to the nipple does not come into contact with the second compression plate while the breast is compressed. The ultrasound transducer array moves along the path to scan the breast. The ultrasound transducer array is disposed adjacent to the second plate on a side opposite to the breast with respect to the second compression plate. The ultrasound transducer array moves along the path to acquire image data representing the breast. In the step of acquiring the image data, image data of one or both of a portion of the breast close to the chest wall and a portion of the breast immediately behind the nipple that does not come into contact with the second compression plate is acquired by operating a direction of an electronic beam using the ultrasound transducer array.

Meanwhile, in a case where an ultrasound image is captured by transmitting and receiving an ultrasound beam through the compression plate in a state where the breast is compressed by the compression plate, due to a difference in acoustic impedance, multiple reflections occur between a front surface and a back surface of the compression plate, and as a result, an artifact occurs in the captured ultrasound image. It goes without saying that this artifact is a major obstacle in performing ultrasound diagnosis by interpreting the ultrasound image.

Therefore, it is desired to suppress this artifact as much as possible, but in the technologies disclosed in each of JP2005-125080A and JP2008-514264A, the artifact is not considered, and thus there is a problem that the artifact cannot necessarily be effectively suppressed.

### SUMMARY

The present disclosure provides an image processing apparatus, a medical image acquisition apparatus, and a program that can effectively suppress an artifact caused by multiple reflections of an ultrasound beam in a compression plate in a case of performing ultrasound imaging of a breast through the compression plate, as compared with the related art.

According to a first aspect of the present disclosure, there is provided an image processing apparatus comprising: a processor, in which the processor is configured to: create a steered transmission/reception image that is an image of a breast obtained by an ultrasound beam which is transmitted and received, via a compression plate, by beam steering with a predetermined steering angle using an ultrasound probe and a non-steered transmission/reception image that is an image of the breast obtained by the ultrasound beam which is transmitted and received, via the compression plate, by non-beam steering using the ultrasound probe, in a state where the breast is compressed by the compression plate; and create a compound image using the created steered transmission/reception image and the created non-steered transmission/reception image, the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and the non-steered transmission/reception image is an image created by using the frequency component as the main frequency component.

The term “main frequency component” used herein means a frequency component that occupies 50% or more of an amplitude of the entire reflected wave. In addition, in the first aspect, the reason why the frequency component in the range in which the amplitude of the artifact caused by the sidelobe of the ultrasound beam is half or less of the amplitude of the ultrasound beam transmitted is applied as the main frequency component is as follows. That is, in a case where the amplitude of the artifact caused by the sidelobe of the ultrasound beam is within the range, a signal-to-noise (S/N) ratio is sufficiently high, and the signal information of the steered transmission/reception image can be sufficiently visually recognized. Therefore, even in a case where the non-steered transmission/reception image is combined with the steered transmission/reception image, the effect of reducing the influence of the artifact caused by the steered transmission/reception image and the clarity as the overall diagnostic image can be secured.

According to a second aspect of the present disclosure, in the image processing apparatus according to the first aspect, the processor is configured to create the compound image in a harmonic mode or a compound harmonic mode.

According to a third aspect of the present disclosure, in the image processing apparatus according to the second aspect, in a case where the compound image is created in the compound harmonic mode, the processor is configured to: increase a weight of a harmonic component for a shallow part including a portion of multiple reflections of the ultrasound beam, as compared with a weight of a fundamental wave component; and increase the weight of the fundamental wave component as a depth increases from the shallow part to a deep part.

According to a fourth aspect of the present disclosure, in the image processing apparatus according to the first aspect, the processor is configured to change a setting to create the compound image in a case where an imaging mode using the compression plate is selected.

According to a fifth aspect of the present disclosure, in the image processing apparatus according to the first aspect, the processor is configured to change a setting to create the compound image in a case where an image corresponding to the compression plate is recognized in at least one of the steered transmission/reception image or the non-steered transmission/reception image.

According to a sixth aspect of the present disclosure, in the image processing apparatus according to the first aspect, the processor is configured to change a setting to create the compound image in a case where the compression by the compression plate is ended.

According to a seventh aspect of the present disclosure, in the image processing apparatus according to the first aspect, the processor is configured to create the compound image by changing a ratio of weights of the steered transmission/reception image and the non-steered transmission/reception image depending on a depth of multiple reflections of the ultrasound beam.

According to an eighth aspect of the present disclosure, in the image processing apparatus according to the first aspect, the processor is configured to create the compound image by not using a reflected wave corresponding to between a front surface that is a surface of the compression plate on a side opposite to the breast and a back surface that is a surface of the compression plate on the side of the breast, increasing a weight of the steered transmission/reception image as compared with a weight of the non-steered transmission/reception image from the back surface of the compression plate to a depth of multiple reflections of the ultrasound beam, and increasing the weight of the non-steered transmission/reception image as the depth increases.

According to a ninth aspect of the present disclosure, in the image processing apparatus according to the first aspect, a sound speed of the ultrasound beam is a sound speed between a sound speed of the breast and a sound speed of the compression plate.

According to a tenth aspect of the present disclosure, in the image processing apparatus according to the first aspect, the main frequency component is a frequency component of 8 MHz or less, and the steering angle is an angle of 20 degrees or less.

According to an eleventh aspect of the present disclosure, in the image processing apparatus according to the first aspect, the main frequency component is a frequency component of 9 MHz or less, and the steering angle is an angle of 15 degrees or less.

According to a twelfth aspect of the present disclosure, in the image processing apparatus according to the first aspect, the main frequency component is a frequency component of 11 MHz or less, and the steering angle is an angle of 10 degrees or less.

In addition, according to a thirteenth aspect of the present disclosure, there is provided a medical image acquisition apparatus comprising: the image processing apparatus according to the first aspect; and a display unit that displays the compound image created by the image processing apparatus.

Further, according to a fourteenth aspect of the present disclosure, there is provided a program causing a computer to execute a process comprising: creating a steered transmission/reception image that is an image of a breast via a compression plate by beam steering with a predetermined steering angle using an ultrasound probe and a non-steered transmission/reception image that is an image of the breast via the compression plate by non-beam steering using the ultrasound probe, in a state where the breast is compressed by the compression plate; and creating a compound image using the created steered transmission/reception image and the created non-steered transmission/reception image, in which the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and the non-steered transmission/reception image is an image created by using the frequency component as the main frequency component.

According to the present disclosure, it is possible to effectively suppress an artifact caused by multiple reflections of an ultrasound beam in a compression plate in a case of performing ultrasound imaging of a breast through the compression plate, as compared with the related art.

### BRIEF DESCRIPTION OF THE DRAWINGS

FIG. 1 is a diagram showing an example of a schematic configuration of an image generation system according to an embodiment.

FIG. 2 is a side view showing an example of an appearance of the image generation apparatus according to the embodiment.

FIG. 3 is a three-view showing an example of a schematic configuration of a compression member according to the embodiment.

FIG. 4 is a three-view showing an example of the schematic configuration of the compression member according to the embodiment.

FIG. 5 is a block diagram showing an example of a hardware configuration of a console according to the embodiment.

FIG. 6 is a block diagram showing an example of a functional configuration of the console according to the embodiment.

FIG. 7 is a graph for describing a main frequency component and a steering angle according to the embodiment, and is a graph showing directivity of an element of a specification of an ultrasound probe for a mammary gland.

FIG. 8 is a diagram showing an example of a harmonic component of an ultrasound image according to the embodiment.

FIG. 9 is a diagram showing an example of a fundamental wave component of the ultrasound image according to the embodiment.

FIG. 10 is a flowchart illustrating an example of image processing according to the embodiment.

FIG. 11 is a diagram showing an example of an initial screen according to the embodiment.

FIG. 12 is a schematic diagram for describing a method of creating a compound image according to the embodiment.

FIG. 13 is a diagram showing an example of a compound image display screen according to the embodiment.

FIG. 14 is a diagram showing another example of the compound image display screen according to the embodiment.

FIG. 15 is a diagram showing another example of the compound image display screen according to the embodiment.

### DETAILED DESCRIPTION

Hereinafter, description regarding embodiments of the present disclosure will be made with reference to the drawings.

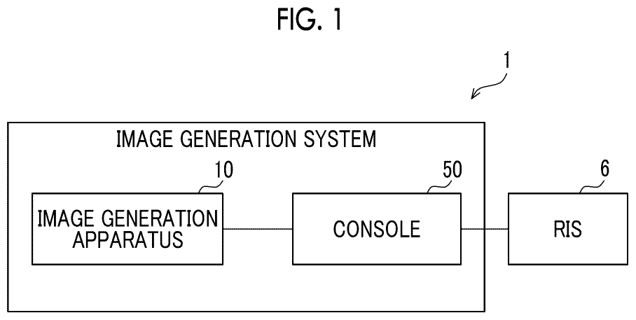

First, a configuration of an image generation system 1 to which a technique of the present disclosure is applied will be described with reference to FIG. 1. FIG. 1 is a diagram showing an example of a schematic configuration of the image generation system 1 according to the present embodiment.

As shown in FIG. 1, the image generation system 1 comprises an image generation apparatus 10 and a console 50. The image generation apparatus 10 and the console 50, and the console 50 and an external radiology information system (RIS) 6 are configured to be connected to each other via a wired or wireless network.

In the image generation system 1 according to the present embodiment, the console 50 acquires an imaging order or the like from the RIS 6, and controls the image generation apparatus 10 in accordance with the imaging order, an instruction from the user, and the like. The image generation apparatus 10 irradiates a breast in a compressed state between the imaging table 16 and a compression member 40 as a compression plate of the technology of the present disclosure, which will be described below, with radiation R to capture a radiation image. In addition, the image generation apparatus 10 acquires an ultrasound image of the breast put into the compressed state via the compression member 40. The image generation system 1 corresponds to a medical image acquisition apparatus in the technology of the present disclosure.

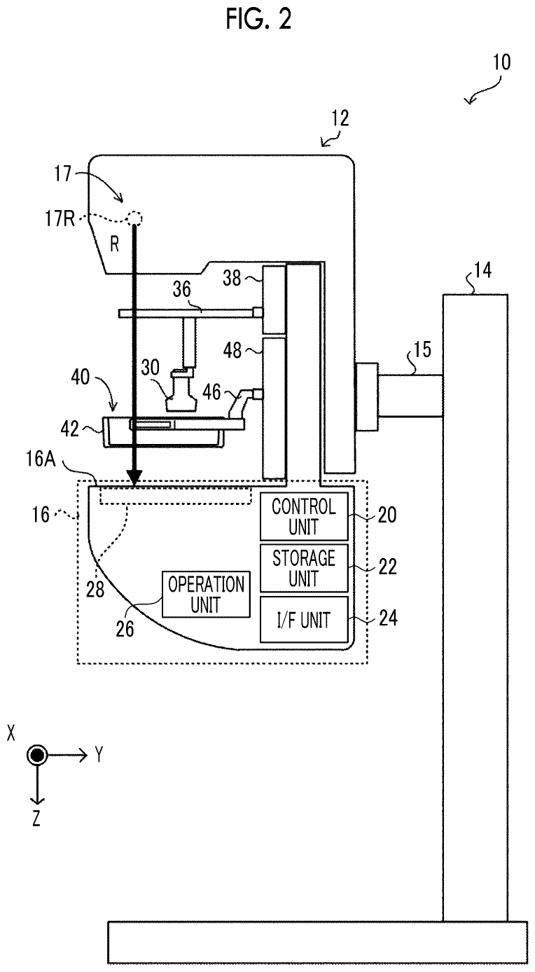

Next, a description of a schematic configuration of the image generation apparatus 10 according to the present embodiment will be made with reference to FIG. 2. FIG. 2 is a side view showing an example of an appearance of the image generation apparatus 10 according to the present embodiment, and is a view in a case where the image generation apparatus 10 is viewed from a right side of a subject. As shown in FIG. 2, the image generation apparatus 10 comprises a radiation source 17R, a radiation detector 28, the imaging table 16 disposed between the radiation source 17R and the radiation detector 28, the compression member 40 that compresses the breast between the compression member 40 and the imaging table 16, and an ultrasound probe 30. In the image generation apparatus 10, a user, such as a doctor or a technician, positions the breast of the subject on an imaging surface 16A of the imaging table 16.

The image generation apparatus 10 comprises an arm part 12, a base 14, and a shaft part 15. The arm part 12 is movably held in the vertical direction (Z direction) by the base 14. The shaft part 15 connects the arm part 12 to the base 14. The arm part 12 is rotatable relative to the base 14, using the shaft part 15 as a rotation axis. In addition, the arm part 12 may be relatively rotatable with respect to the base 14 with the shaft part 15 as the rotation axis separately between an upper part comprising a radiation emitting unit 17 and a lower part comprising the imaging table 16.

The arm part 12 comprises the radiation emitting unit 17 and the imaging table 16. The radiation emitting unit 17 comprises the radiation source 17R, and is configured to change an irradiation field of radiation (for example, X-rays) emitted from the radiation source 17R. The change of the irradiation field may be performed, for example, by the user operating an operation unit 26 or by a control unit 20 in accordance with a type of the attached compression member 40.

The imaging table 16 comprises the control unit 20, a storage unit 22, an interface (I/F) unit 24, the operation unit 26, and the radiation detector 28. The control unit 20 controls an overall operation of the image generation apparatus 10 in accordance with the control of the console 50. The control unit 20 comprises a central processing unit (CPU), a read only memory (ROM), a random access memory (RAM), and the like (not shown). The ROM stores in advance various programs, including a program for controlling the generation of the radiation image and the ultrasound image, which are executed by the CPU. The RAM temporarily stores various data.

Data of the radiation image and the ultrasound image, various types of other information, and the like are stored in the storage unit 22. The storage unit 22 is realized by, for example, a storage medium, such as a hard disk drive (HDD), a solid state drive (SSD), and a flash memory.

The I/F unit 24 performs communication of various types of information with the console 50 through wired communication or wireless communication. Specifically, the I/F unit 24 receives information related to the control of the image generation apparatus 10 from the console 50. In addition, the I/F unit 24 transmits the data of the radiation image and the ultrasound image to the console 50.

The operation unit 26 is a part that is provided on the imaging table 16 or the like and that is operable by the user with a hand, a foot, or the like, and is, for example, a switch, a button, or a touch panel.

The radiation detector 28 is disposed in the imaging table 16, detects the radiation R transmitted through the breast and the imaging table 16, generates the radiation image based on the detected radiation R, and outputs image data indicating the generated radiation image. It should be noted that a type of the radiation detector 28 is not particularly limited and may be, for example, an indirect conversion type radiation detector that converts the radiation R into light and converts the converted light into a charge, or a direct conversion type radiation detector that directly converts the radiation R into a charge.

A probe unit 38 and a compression unit 48 are connected to the arm part 12. A support part 36 that attachably and detachably supports the ultrasound probe 30 is attached to the probe unit 38. The support part 36 (ultrasound probe 30) is moved in the vertical direction and the horizontal direction (X direction, Y direction, and Z direction) by a driving unit (not shown) provided in the probe unit 38. The support part 36 is preferably formed of a material that transmits the radiation R.

The ultrasound probe 30 is used for obtaining the ultrasound image of the breast put into the compressed state by the compression member 40, is disposed between the radiation source 17R and the compression member 40, irradiates the breast with ultrasound waves via the compression member 40, and receives reflected waves from the breast.

Specifically, the ultrasound probe 30 comprises an ultrasound transducer array. The ultrasound transducer array has a configuration in which a plurality of ultrasound transducers are arranged one-dimensionally or two-dimensionally. The ultrasound transducer may be constituted, for example, by forming electrodes at both ends of a piezoelectric body such as a piezoelectric ceramic represented by lead zirconate titanate (PZT), a piezoelectric single crystal represented by lead magnesium niobate-lead titanate (PMN-PT), and a polymer piezoelectric element represented by polyvinylidene difluoride (PVDF). In addition, for example, the ultrasound transducer may be a capacitive micro-machined ultrasound transducer (CMUT).

In addition, a plurality of types of the ultrasound probes 30 different from each other may be interchangeably attached to the image generation apparatus 10. Specifically, in accordance with a physique of the subject (for example, a size of the breast), a tissue composition of the breast (for example, a fat mass and a mammary gland mass), a type of imaging (for example, magnified imaging and spot imaging), and the like, the ultrasound probes 30 each having different performances and dimensions may be attached. For example, a linear probe having a center frequency of about 7.5 MHz (for superficial use or the like), a convex probe having a center frequency of about 3.5 MHz (for abdomen or the like), a sector probe having a center frequency of about 2.5 MHz (for heart or the like), and the like may be used.

A support part 46 that supports the compression member 40 is attachably and detachably attached to the compression unit 48. The support part 46 (compression member 40) is moved in the up-down direction (Z direction) by a driving unit (not shown) provided in the compression unit 48.

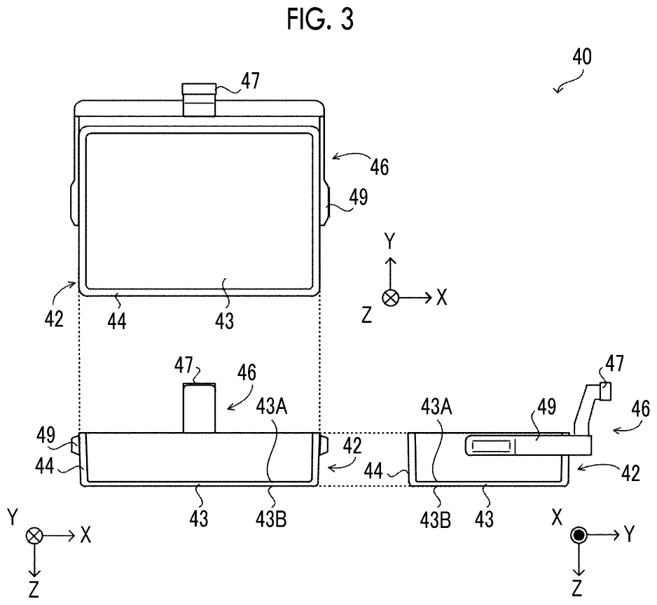

The compression member 40 is disposed between the radiation source 17R and the imaging table 16 and sandwiches the breast between the compression member 40 and the imaging table 16 to put the breast into a compressed state. FIG. 3 shows a three-plane diagram of an example of the compression member 40. The three-plane diagram of FIG. 3 includes a top view of the compression member 40 as viewed from above (radiation emitting unit 17 side), a side view thereof as viewed from a subject side, and a side view thereof as viewed from a right side of the subject. As shown in FIG. 3, the compression member 40 includes a compression part 42 and the support part 46.

The support part 46 includes an attachment portion 47 and an arm 49. The attachment portion 47 attaches the compression member 40 to the image generation apparatus 10, specifically, the driving unit of the compression unit 48. The arm 49 supports the compression part 42.

The compression part 42 includes a bottom portion 43 formed to be substantially flat and surrounded by a wall portion 44 having a substantially uniform height and has a recessed cross-sectional shape. The compression part 42 is preferably formed of an optically transparent or translucent material in order to perform positioning and confirmation of a compressed state in compression of the breast. In addition, the compression part 42 is preferably formed of a material having excellent transmittance of the radiation R and ultrasonic waves. In addition, the compression part 42 is preferably formed of, for example, a material excellent in strength such as drop strength and compression strength.

As such a material, for example, a resin such as polymethylpentene (PMP), polycarbonate (PC), acryl, polypropylene (PP), and polyethylene terephthalate (PET) can be used. In particular, in the polymethylpentene, acoustic impedance, which affects transmissivity and reflectivity of ultrasonic waves, is close to that of a human body (breast) as compared with other materials, and a proportion of noise on the ultrasound image can be reduced. Therefore, the polymethylpentene is suitable as the material of the compression part 42.

In addition, a plurality of types of the compression members 40 different from each other may be interchangeably attached to the image generation apparatus 10. Specifically, in accordance with a physique of the subject (for example, a size of the breast), a tissue composition of the breast (for example, a fat mass and a mammary gland mass), a type of imaging (for example, magnified imaging and spot imaging), and the like, the compression members 40 having different materials, sizes, and shapes from each other may be attached. For example, a compression member according to the size of the breast, a compression member for axillary imaging, a compression member for magnified imaging, a compression member for so-called spot imaging that captures a radiation image of only a region where a lesion exists, and the like may be used. That is, the compression member 40 is not limited to the compression member that compresses the entire breast, and may have a smaller size than the breast to compress a part of the breast.

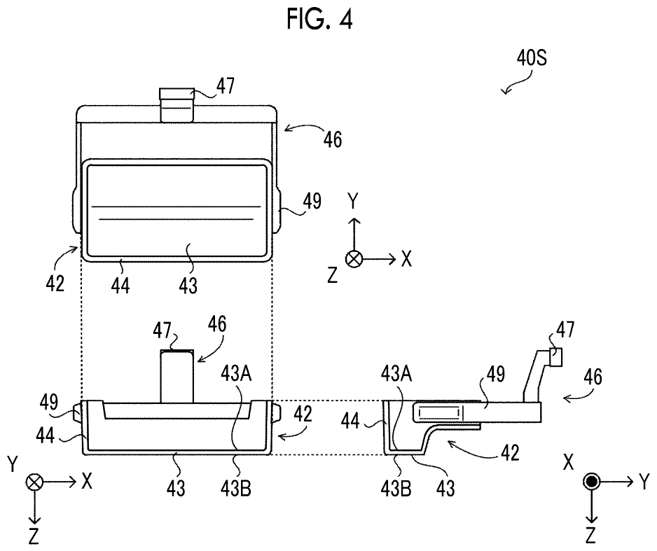

FIG. 4 shows a three-view of the compression member 40S for a small breast as an example of another form different from the compression member 40 of FIG. 3. The three-view of FIG. 4 includes a top view of the compression member 40S as viewed from above (the radiation emitting unit 17 side), a side view thereof as viewed from the subject side, and a side view thereof as viewed from the right side of the subject. The compression member 40S includes the compression part 42 and the support part 46, similarly to the compression member 40 in FIG. 3. In the compression member 40S, the bottom portion 43 is not flat, and the attachment portion 47 side is higher than a chest wall side (side away from attachment portion 47). In addition, a height of the wall portion 44 is not uniform, and a height of a part of the chest wall side is lower than a height of other portions. Due to such a shape, the compression member 40S can easily perform positioning and compression even in a small breast.

As described above, in the image generation apparatus 10, at least one of the compression member 40 for putting the breast into the compressed state or the ultrasound probe 30 for acquiring the ultrasound image can be attached and detached. In this case, the image generation apparatus 10 may detect the types of the compression member 40 and the ultrasound probe 30 that are attached.

For example, the attachment portion 47 of the compression member 40 may be provided with a plurality of pins having different dispositions for each type of the compression member 40 as identification information, and the identification information may be read by a sensor (for example, a photointerruptor) capable of detecting the disposition of the pins provided in the compression unit 48. Further, for example, a marker (for example, a bar code, a two-dimensional code, or the like) corresponding to the type of the compression member 40 may be provided at any position of the compression member 40 as identification information, and the identification information may be read by a sensor (for example, a charge coupled device (CCD) sensor or the like) capable of detecting the marker.

Further, for example, a radio frequency identification (RFID) tag having identification information corresponding to the type of the compression member 40 may be provided at any position of the compression member 40 and the identification information may be read by an RFID reader capable of reading the RFID tag. Further, for example, a weight of each type of the compression member 40 and the identification information may be stored in the storage unit 22 in advance in association with each other, a weight of the attached compression member 40 may be measured by a sensor capable of detecting the weight, and the identification information (type of the compression member 40) may be specified based on a measured value.

Similarly, for the ultrasound probe 30, the type of the attached ultrasound probe 30 may be identified in accordance with, for example, a pin, a marker, an RFID tag, a weight, or the like.

In addition, the image generation apparatus 10 detects a compression pressure applied to the breast by the compression member 40. For example, in a case where the compression member 40 compresses the breast, a reaction force equal to the compression pressure is applied to the driving unit of the compression member 40. Using this, a strain gauge (for example, a load cell or the like) for detecting the reaction force applied to the driving unit may be provided in the compression unit 48, and the reaction force detected by the strain gauge may be detected as the compression pressure. Further, for example, the compression pressure may be detected by using a semiconductor pressure sensor, a capacitive pressure sensor, and the like. Further, for example, various sensors for detecting the compression pressure may be provided on the compression member 40 side instead of the compression unit 48 side.

In addition, the image generation apparatus 10 detects a distance between the imaging surface 16A of the imaging table 16 and a contact surface 43B of the bottom portion 43 of the compression part 42 in the compression member 40 in a case where the compression member 40 compresses the breast (hereinafter, referred to as “compression thickness”). For example, a distance sensor using laser light, ultrasound waves, or the like may be used to detect the compression thickness. In addition, a form may be adopted in which the compression thickness is detected from a position of the support part 46 in the vertical direction.

In addition, an upper surface 43A and/or a breast contact surface 43B of the bottom portion 43 of the compression member 40 may be coated with a gel-like or liquid medium having ultrasound transmittance. As such a medium, for example, a known jelly for an ultrasound examination, which has the acoustic impedance close to the acoustic impedance of the human body (breast), can be applied. That is, the image generation apparatus 10 may acquire the ultrasound image of the breast put into the compressed state by the compression member 40 in a state of being applied with the gel-like or liquid-like medium having the ultrasound transmittance, via the compression member 40. In this case, it is possible to suppress entrance of air into an interface between an ultrasound radiation surface of the ultrasound probe 30 and the upper surface 43A and/or an interface between the contact surface 43B and the breast, and it is possible to reduce a difference in acoustic impedance at each interface, so that the proportion of the noise applied to the ultrasound image can be reduced.

In addition, the method of imaging the breast via the image generation apparatus 10 is not particularly limited. For example, cranio-caudal (CC) imaging, medio-lateral oblique (MLO) imaging, magnified imaging and spot imaging for imaging a part of the breast, and the like may be performed. The CC imaging is a method of imaging the breast in a compressed state by sandwiching the breast between the imaging table 16 and the compression member 40 in the up-down direction (Z direction). The MLO imaging is a method of imaging the breast in a compressed state, including an axillary portion, by sandwiching the breast between the imaging table 16 and the compression member 40 in a tilted state where a rotation angle of the arm part 12 with respect to the base 14 is 45 degrees or more and less than 90 degrees.

In addition, for example, the image generation apparatus 10 may perform tomosynthesis imaging. In the tomosynthesis imaging, the radiation R is emitted from each of a plurality of irradiation positions having different irradiation angles toward the breast by the radiation source 17R, and a plurality of radiation images of the breast are captured. That is, in the tomosynthesis imaging, the imaging is performed by changing a rotation angle of the radiation emitting unit 17 with respect to the base 14 while angles of the imaging table 16, the compression member 40, the breast, and the like are fixed.

In addition, in the image generation apparatus 10, the breast of the subject may be positioned not only in a state where the subject is standing (standing state) but also in a state where the subject is sitting on a chair, a wheelchair, or the like (sitting state).

The console 50 sets an upper limit value of the compression pressure applied to the breast by the compression member 40 in accordance with the type of the compression member 40 attached to the image generation apparatus 10. In addition, the console 50 controls the image generation apparatus 10 to acquire the radiation image in accordance with the imaging order acquired from the RIS 6, the instruction from the user, and the like. In addition, the console 50 controls a position of the ultrasound probe 30 so that the ultrasound image can be acquired in accordance with a position of a region of interest included in the radiation image captured in the image generation apparatus 10.

As described above, the image generation apparatus 10 according to the present embodiment is provided with the probe unit 38 between the radiation emitting unit 17 and the compression unit 48, and can acquire both the radiation image and the ultrasound image while the breast is put into the compressed state by the compression member 40. As a result, registration between these images is facilitated, and a differential determination ability of the lesion can be improved by displaying both images in a superimposed manner.

Next, the console 50 according to the present embodiment will be described.

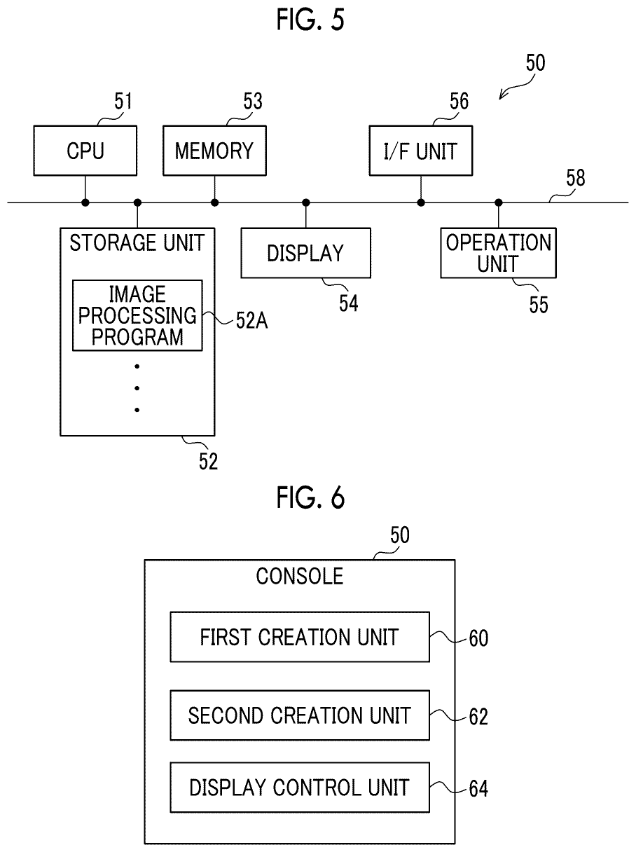

First, an example of a hardware configuration of the console 50 will be explained with reference to FIG. 5. As shown in FIG. 5, the console 50 includes a CPU 51, a non-volatile storage unit 52, and a memory 53 as a temporary storage area. In addition, the console 50 includes a display 54 as a display unit such as a liquid crystal display, an operation unit 55 such as a touch panel, a keyboard, and a mouse, and an I/F unit 56. The I/F unit 56 performs wired or wireless communication with the image generation apparatus 10, the RIS 6, other external apparatuses, and the like. The CPU 51, the storage unit 52, the memory 53, the display 54, the operation unit 55, and the I/F unit 56 are connected to each other via a bus 58 such as a system bus and a control bus, so that various types of information can be exchanged.

The storage unit 52 is implemented by, for example, a storage medium such as an HDD, an SSD, and a flash memory. An image processing program 52A is stored in the storage unit 52. The CPU 51 reads out the image processing program 52A from the storage unit 52, loads the image processing program 52A into the memory 53, and executes the loaded image processing program 52A. As the console 50, for example, a personal computer, a server computer, a smartphone, a tablet terminal, or a wearable terminal can be appropriately applied.

Further, the storage unit 52 stores image data of the radiation image and the ultrasound image created by the data acquired by the image generation apparatus 10, and various other types of information. The image data of the radiation image and the ultrasound image may be stored in association with at least one of an imaging order or imaging information. The imaging information may be, for example, at least one of subject information and an imaging item that are included in the imaging order, imaging person information indicating an imaging person (for example, a user such as a doctor or a technician) who performed the imaging, or date and time information indicating date and time when the imaging is performed.

Next, an example of a functional configuration of the console 50 will be described with reference to FIG. 6. As shown in FIG. 6, the console 50 includes a first creation unit 60, a second creation unit 62, and a display control unit 64. The CPU 51 executes the image processing program 52A to function as the first creation unit 60, the second creation unit 62, and the display control unit 64.

The first creation unit 60 according to the present embodiment creates a steered transmission/reception image that is an image of a breast via a compression member 40 by beam steering with a predetermined steering angle using an ultrasound probe 30 and a non-steered transmission/reception image that is an image of the breast via the compression member 40 by non-beam steering using the ultrasound probe 30, in a state where the breast is compressed by the compression member 40. Then, the second creation unit 62 according to the present embodiment creates a compound image using the steered transmission/reception image and the non-steered transmission/reception image created by the first creation unit 60. The angle in the steering angle used herein means an angle with respect to an emission direction of the ultrasound beam from the ultrasound probe 30.

In the present embodiment, as the steered transmission/reception image, an image created by using, as a main frequency component, a frequency component in a range in which an amplitude of an artifact caused by a sidelobe of the ultrasound beam is half or less of an amplitude of the ultrasound beam transmitted to create the steered transmission/reception image is applied, and as the non-steered transmission/reception image, an image created by using the frequency component as the main frequency component is applied.

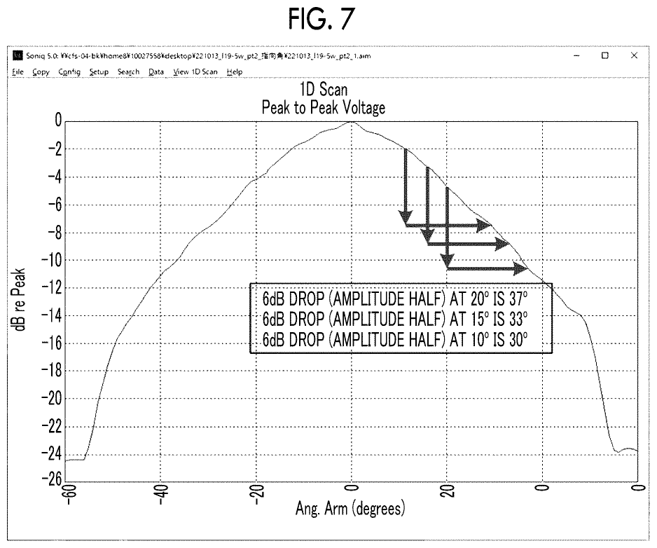

Here, the principle of the technology of the present disclosure will be described with reference to FIG. 7. FIG. 7 is a graph for describing a main frequency component and a steering angle according to the present embodiment, and is a graph showing directivity of an element of a specification of an ultrasound probe for a mammary gland.

A sidelobe emission angle (hereinafter, referred to as a “sidelobe angle”) θm in a case of beam steering is represented by the following expression (1). Here, θs represents the steering angle, p represents a pitch of the element (ultrasound transducer) of the ultrasound probe 30, and λ represents a wavelength of the ultrasound beam. The angle in the sidelobe angle used herein also means an angle with respect to the emission direction of the ultrasound beam from the ultrasound probe 30.

$$\begin{matrix}[ Expression 1 ] & \\ {θ}_{m} = {sin}^{- 1} ( \frac{λ}{p} - sin {θ}_{s} ) & ( 1 )\end{matrix}$$

Here, in general, as described in, for example, <URL:https://www.ndk.com/jp/products/ultrasound/probe/linear_probe/#020> on the Internet, a specification of the ultrasound probe used for diagnosing the mammary gland is such that the pitch p of the element is about 0.2 mm, and the frequency is about 5 to 15 MHz.

On the other hand, in general, in a case where a transmission and reception direction of the ultrasound beam deviates from a vertical direction of the element, the amplitude is reduced. As an example, as shown in FIG. 7, the amplitude of the sound wave transmitted in a direction of 20 degrees is twice (6 dB difference) the amplitude of the sound wave transmitted in a direction of 37 degrees. Similarly, the amplitude of the sound wave transmitted in a direction of 15 degrees is twice the amplitude of the sound wave transmitted in a direction of 33 degrees, and the amplitude of the sound wave transmitted in a direction of 10 degrees is twice the amplitude of the sound wave transmitted in a direction of 30 degrees.

Therefore, the developers of the technology of the present disclosure obtained a relationship between each frequency component in a range in which the pitch p is 0.2 mm and the transmission frequency f of the ultrasound beam is 5 MHz to 15 MHz, and the steering angle θs and the sidelobe angle θm by using the expression (1), and summarized the results as shown in Tables 1 to 3. Table 1 is a table for obtaining the main frequency component in a case where the amplitude of the artifact caused by the sidelobe is half or less of the amplitude of the ultrasound beam in a case where the steering angle θs is transmitted as 20 degrees or less. In addition, Table 2 is a table for obtaining the main frequency component in a case where the amplitude of the artifact caused by the sidelobe is half or less of the amplitude of the ultrasound beam in a case where the steering angle θs is transmitted as 15 degrees or less. Further, Table 3 is a table for obtaining the main frequency component in a case where the amplitude of the artifact caused by the sidelobe is half or less of the amplitude of the ultrasound beam in a case where the steering angle θs is transmitted as 10 degrees or less.

TABLE 1

| f (MHz) | p (mm) | λ (mm) | λ/p | θ m (deg) | sin (θs) | θs (deg) |
|---|---|---|---|---|---|---|
| 5.00 | 0.20 | 0.31 | 1.54 | 20.00 | 1.20 | |
| 6.00 | 0.20 | 0.26 | 1.28 | 20.00 | 0.94 | 70.27 |
| 7.00 | 0.20 | 0.22 | 1.10 | 20.00 | 0.76 | 49.29 |
| 8.00 | 0.20 | 0.19 | 0.96 | 20.00 | 0.62 | 38.35 |
| 9.00 | 0.20 | 0.17 | 0.86 | 20.00 | 0.51 | 30.90 |
| 10.00 | 0.20 | 0.15 | 0.77 | 20.00 | 0.43 | 25.34 |
| 11.00 | 0.20 | 0.14 | 0.70 | 20.00 | 0.36 | 20.98 |
| 12.00 | 0.20 | 0.13 | 0.64 | 20.00 | 0.30 | 17.44 |
| 13.00 | 0.20 | 0.12 | 0.59 | 20.00 | 0.25 | 14.49 |
| 14.00 | 0.20 | 0.11 | 0.55 | 20.00 | 0.21 | 12.00 |
| 15.00 | 0.20 | 0.10 | 0.51 | 20.00 | 0.17 | 9.86 |

TABLE 2

| f (MHz) | p (mm) | λ (mm) | λ/p | θ m (deg) | sin (θs) | θs (deg) |
|---|---|---|---|---|---|---|
| 5.00 | 0.20 | 0.31 | 1.54 | 15.00 | 1.28 | |
| 6.00 | 0.20 | 0.26 | 1.28 | 15.00 | 1.02 | |
| 7.00 | 0.20 | 0.22 | 1.10 | 15.00 | 0.84 | 57.27 |
| 8.00 | 0.20 | 0.19 | 0.96 | 15.00 | 0.70 | 44.72 |
| 9.00 | 0.20 | 0.17 | 0.86 | 15.00 | 0.60 | 36.64 |
| 10.00 | 0.20 | 0.15 | 0.77 | 15.00 | 0.51 | 30.74 |
| 11.00 | 0.20 | 0.14 | 0.70 | 15.00 | 0.44 | 26.18 |
| 12.00 | 0.20 | 0.13 | 0.64 | 15.00 | 0.38 | 22.51 |
| 13.00 | 0.20 | 0.12 | 0.59 | 15.00 | 0.33 | 19.48 |
| 14.00 | 0.20 | 0.11 | 0.55 | 15.00 | 0.29 | 16.93 |
| 15.00 | 0.20 | 0.10 | 0.51 | 15.00 | 0.25 | 14.74 |

TABLE 3

| f (MHz) | p (mm) | λ (mm) | λ/p | θ m (deg) | sin (θs) | θs (deg) |
|---|---|---|---|---|---|---|
| 5.00 | 0.20 | 0.31 | 1.54 | 10.00 | 1.37 | |
| 6.00 | 0.20 | 0.26 | 1.28 | 10.00 | 1.11 | |
| 7.00 | 0.20 | 0.22 | 1.10 | 10.00 | 0.93 | 67.87 |
| 8.00 | 0.20 | 0.19 | 0.96 | 10.00 | 0.79 | 52.08 |
| 9.00 | 0.20 | 0.17 | 0.86 | 10.00 | 0.68 | 42.99 |
| 10.00 | 0.20 | 0.15 | 0.77 | 10.00 | 0.60 | 36.61 |
| 11.00 | 0.20 | 0.14 | 0.70 | 10.00 | 0.53 | 31.76 |
| 12.00 | 0.20 | 0.13 | 0.64 | 10.00 | 0.47 | 27.91 |
| 13.00 | 0.20 | 0.12 | 0.59 | 10.00 | 0.42 | 24.75 |
| 14.00 | 0.20 | 0.11 | 0.55 | 10.00 | 0.38 | 22.11 |
| 15.00 | 0.20 | 0.10 | 0.51 | 10.00 | 0.34 | 19.86 |

Then, the developers of the technology of the present disclosure obtained the transmission frequency of the ultrasound beam in which the amplitude of the artifact caused by the sidelobe is half or less of the amplitude of the transmitted ultrasound beam by using the values (the values of 37 degrees, 33 degrees, and 30 degrees) described with reference to FIG. 7 as the threshold values. The transmission frequency is a frequency corresponding to a portion subjected to masking in Tables 1 to 3, and the frequency in a case where the steering angle θs is set to 20 degrees or less is 8 MHz or less. Similarly, the frequency in a case where the steering angle θs is set to 15 degrees or less is 9 MHz or less, and the frequency in a case where the steering angle θs is set to 10 degrees or less is 11 MHz or less.

Therefore, in the console 50 according to the present embodiment, the frequency component of 8 MHz or less is applied as the main frequency component, and 20 degrees or less is applied as the steering angle θs. However, the present disclosure is not limited to this form, and a form in which the frequency component of 9 MHz or less is applied as the main frequency component and 15 degrees or less is applied as the steering angle θs may be adopted. Further, a form in which the frequency component of 11 MHz or less is applied as the main frequency component and 10 degrees or less is applied as the steering angle θs may be adopted. By applying any of the above combinations as the combination of the main frequency component and the steering angle θs, the influence of the multiple reflections caused by the sidelobe can be suppressed, and as a result, the artifact caused by the multiple reflections of the ultrasound beam in the compression member 40 can be effectively suppressed.

In addition, the second creation unit 62 according to the present embodiment creates the compound image in a harmonic mode or a compound harmonic mode.

The harmonic mode is a mode in which the image is created by using only the harmonic component, and a tissue harmonic imaging (THI) mode and a contrast harmonic imaging (CHI) mode can be applied, but in the present embodiment, the THI mode is applied.

On the other hand, the compound harmonic mode is a mode in which the image is created by using both the harmonic component and the fundamental wave component, and the images are weighted and added to each other to create the image.

In a case where the compound image is created in the compound harmonic mode, the second creation unit 62 according to the present embodiment increases the weight of the harmonic component for a shallow part including a portion of the multiple reflections of the ultrasound beam, as compared with the weight of the fundamental wave component, and increases the weight of the fundamental wave component as the depth increases from the shallow part to a deep part.

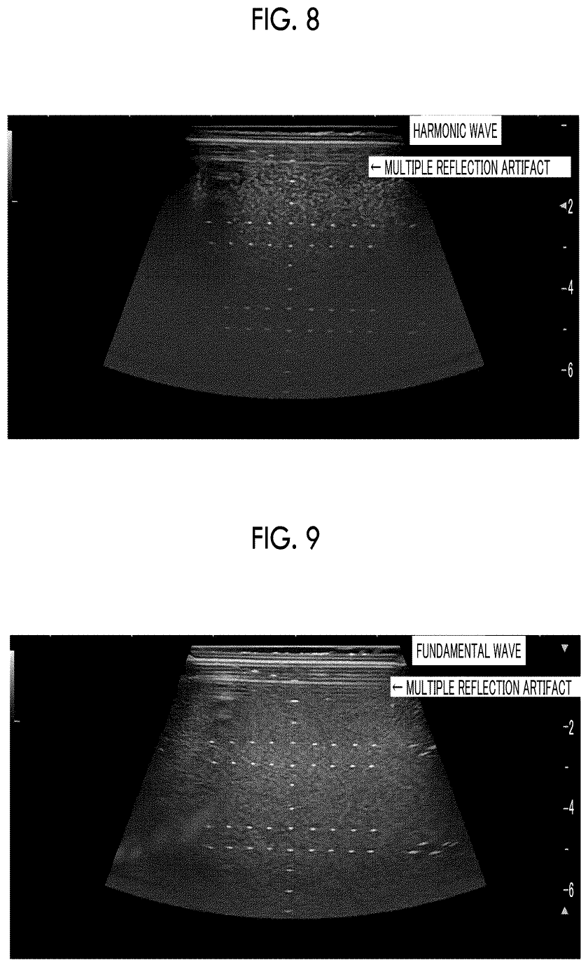

FIG. 8 shows an example of the harmonic component of the ultrasound image according to the present embodiment, and FIG. 9 shows an example of the fundamental wave component of the ultrasound image according to the present embodiment.

As shown in FIGS. 8 and 9, the harmonic component is attenuated as the entire image, while the artifact is small. On the other hand, the fundamental wave component is easily visible in the deep part, but is difficult to see in the shallow part. Therefore, by increasing the weight of the fundamental wave in the deep part in which the harmonic component is generally attenuated in the compound harmonic mode to create the image, a high-definition image can be obtained in the shallow part, and the penetration (reach in the depth direction) in the deep part can be secured.

As a method of creating the image in the compound harmonic mode, there is a method of performing two transmissions and receptions for the harmonic component and for the fundamental wave component to create the image, and a method of creating the image by performing only one transmission and reception by detecting the broadband pulse at the frequency of the harmonic component to create the image and detecting the fundamental wave component at the frequency to create the image and performing weighted addition.

On the other hand, the second creation unit 62 according to the present embodiment changes the setting to create the compound image in a case where the compression by the compression member 40 is ended. As a result, unnecessary ultrasound imaging in a state where the compression by the compression member 40 is not ended can be avoided.

In addition, the second creation unit 62 according to the present embodiment creates the compound image by changing the ratio of the weights of the steered transmission/reception image and the non-steered transmission/reception image according to the depth of the multiple reflections of the ultrasound beam. In particular, the second creation unit 62 according to the present embodiment creates the compound image by not using a reflected wave corresponding to between a front surface that is a surface of the compression member 40 on a side opposite to the breast and a back surface that is a surface of the compression member 40 on the side of the breast, increasing a weight of the steered transmission/reception image as compared with a weight of the non-steered transmission/reception image from the back surface of the compression member 40 to a depth of the multiple reflections of the ultrasound beam, and increasing the weight of the non-steered transmission/reception image as the depth increases.

Further, in the present embodiment, the sound speed of the ultrasound beam is a sound speed between the sound speed in the breast (for example, a sound speed of 1450 m/s set in a case where a preset for the mammary gland is selected) and the sound speed in the compression member 40 (for example, 2097 m/s) (in the present embodiment, 1490 m/s).

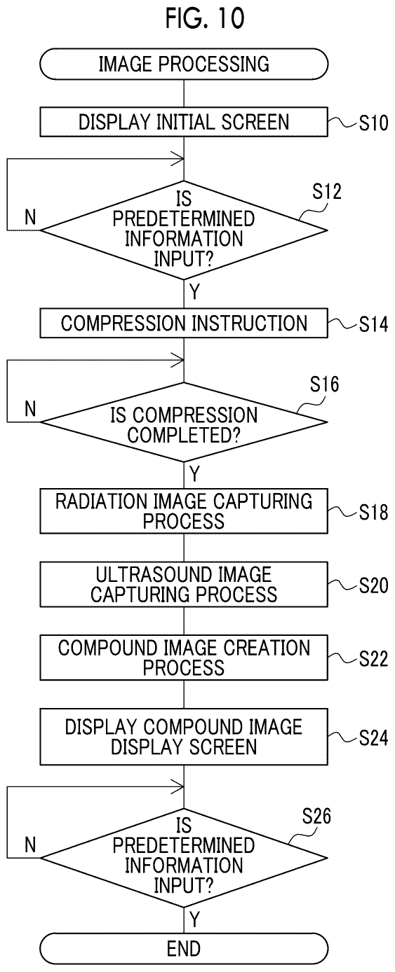

Next, an action of the console 50 according to the present embodiment will be described with reference to FIG. 10. In the console 50, the CPU 51 executes the image processing program 52A to execute the image processing shown in FIG. 10. The image processing is executed, for example, in a case where the user gives an instruction to start the execution via the operation unit 55. Here, a case will be described in which the console 50 acquires information (hereinafter, referred to as “setting information”) indicating the compression pressure and the compression thickness in a case of capturing the radiation image of the breast from the RIS. In addition, here, a case will be described in which the breast (hereinafter, referred to as a “target breast”) of the subject is positioned on the imaging surface 16A of the imaging table 16 by the user such as the doctor and the technician, and an appropriate ultrasound probe 30 is mounted on the probe unit 38.

In step S10, the CPU 51 controls the display 54 to display an initial screen having a predetermined configuration, and in step S12, the CPU 51 waits until predetermined information is input.

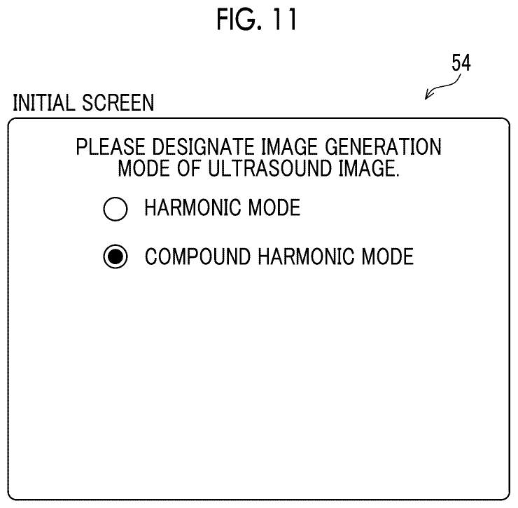

FIG. 11 shows an example of the initial screen according to the present embodiment. As shown in FIG. 11, in the initial screen according to the present embodiment, a message prompting the designation of the image generation mode of the ultrasound image is displayed. In addition, in the initial screen according to the present embodiment, two types of the harmonic mode and the compound harmonic mode described above are displayed in a state where each of the types can be designated as the image generation mode that can be designated.

For example, in a case where the initial screen shown in FIG. 11 is displayed on the display 54, the user designates any image generation mode via the operation part 55. In a case where any one of the image generation modes is designated by the user, the determination in step S12 is “YES”, and the process proceeds to step S14.

In step S14, the CPU 51 gives an instruction to the image generation apparatus 10 such that the compression pressure and the compression thickness by the compression member 40 are set to values indicated by the setting information, so that the CPU gives an instruction to start the compression by the compression member 40 on the target breast. In step S16, the CPU 51 waits until the compression of the target breast by the compression member 40 is completed (compression pressure and compression thickness are set to the values indicated by the setting information). Through the above processing, the compressed state of the target breast by the compression member 40 is the state in a case of capturing each image.

In step S18, the CPU 51 controls the image generation apparatus 10 to capture the radiation image. With this control, the image generation apparatus 10 captures the radiation image of the target breast in which the compressed state by the compression member 40 is set to the state indicated by the setting information, and generates the image data indicating the radiation image obtained by the imaging. As described above, various types of information required for capturing the radiation image in this case are acquired from the RIS.

In step S20, the CPU 51 controls the image generation apparatus 10 to capture the steered transmission/reception image and the non-steered transmission/reception image under a condition corresponding to the mode designated by the user on the initial screen. In response to this control, the image generation apparatus 10 generates the steered transmission/reception image and the non-steered transmission/reception image, and transmits the images to the console 50. In this case, as described above, an angle of 20 degrees or less is applied as the steering angle θs in a case of performing the imaging for generating the steered transmission/reception image.

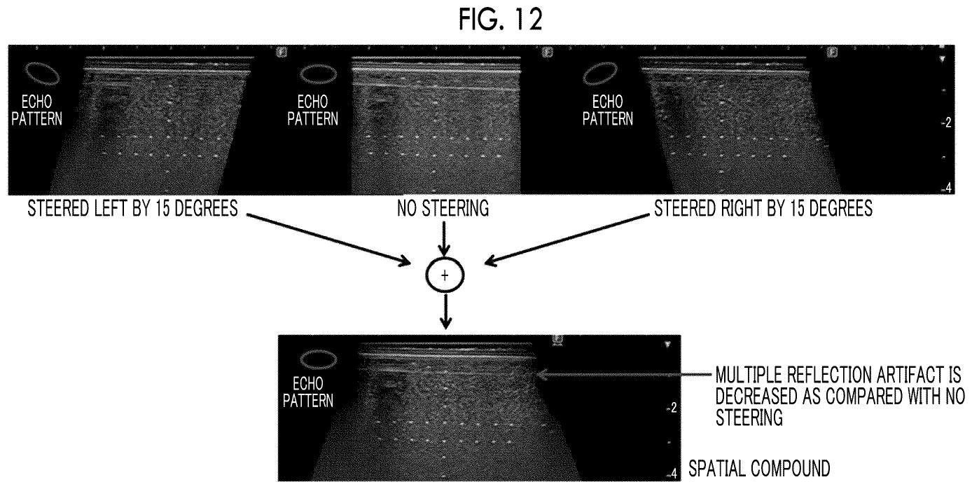

In step S22, the CPU 51 creates the compound image by using the steered transmission/reception image and the non-steered transmission/reception image received from the image generation apparatus 10. FIG. 12 is a schematic diagram for describing a method of creating the compound image according to the present embodiment.

As shown in FIG. 12, in the image processing according to the present embodiment, the compound image is created by performing weighted addition and averaging of the steered transmission/reception image (referred to as “steered” in FIG. 12) with the steering angle of 15 degrees on the left side and 15 degrees on the right side and the non-steered transmission/reception image (referred to as “no steering” in FIG. 12) as described above.

In step S24, the CPU 51 controls the display 54 to display the compound image display screen having a predetermined configuration by using the created compound image, and in step S26, the CPU 51 waits until predetermined information is input.

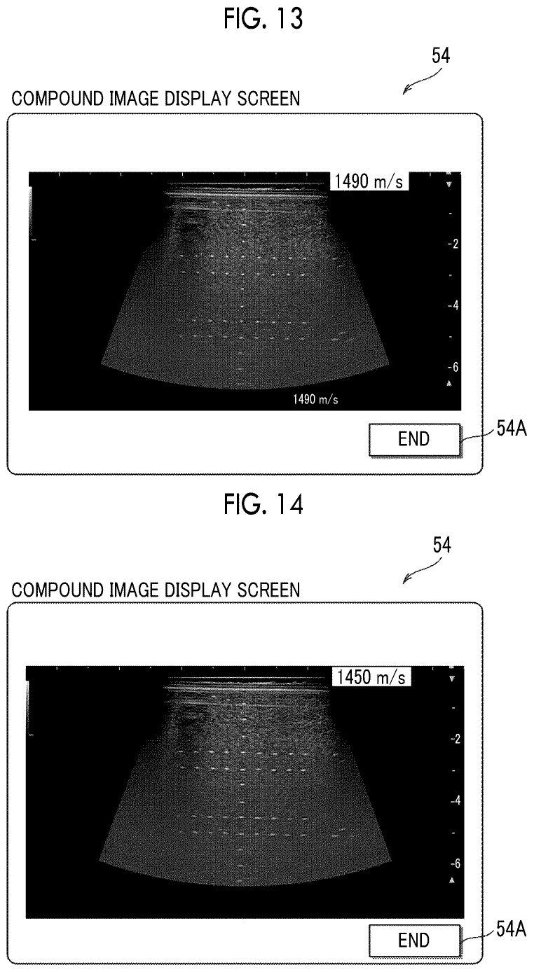

FIG. 13 shows an example of the compound image display screen according to the present embodiment. As shown in FIG. 13, on the compound image display screen according to the present embodiment, the sound speed of the ultrasound beam emitted from the ultrasound probe 30 (in the present embodiment, 1490 m/s) is displayed, and the created compound image is displayed.

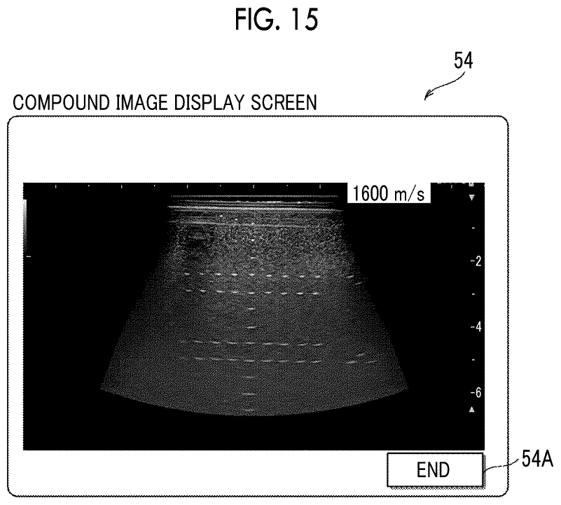

FIGS. 14 and 15 show another example of the compound image display screen according to the present embodiment. FIG. 14 shows the compound image in a case where the sound speed of the ultrasound beam is set to 1450 m/s, and FIG. 15 shows the compound image in a case where the sound speed of the ultrasound beam is set to 1600 m/s, and other conditions are the same as those shown in FIG. 13.

The compound images shown in FIGS. 13 to 15 are captured using a phantom in which a plurality of pins for grasping the resolution of the image created by the imaging are regularly provided, and each pin should have a shape of a true circle in front view.

In a case where the compound images shown in FIGS. 13 to 15 are compared, it can be seen that, as compared with the compound image shown in FIG. 13 (compound image created by setting the sound speed of the ultrasound beam to the sound speed between the sound speed of the breast and the sound speed of the compression member 40), the shapes of the pins in the compound images shown in FIGS. 14 and 15 extend in the lateral direction as the depth increases, and the resolution is low.

As described above, by setting the sound speed of the ultrasound beam to the sound speed between the sound speed of the breast and the sound speed of the compression member 40, the resolution of the compound image can be improved as compared with other cases.

For example, in a case where the compound image display screen shown in FIG. 13 is displayed on the display 54, the user interprets the displayed compound image, and then designates an end button 54A via the operation part 55. In a case where the end button 54A is designated by the user, the determination in step S26 is “YES”, and the present image processing ends.

As described above, with the console 50 as the image processing apparatus according to the present embodiment, the steered transmission/reception image that is the image of the breast via the compression plate by beam steering with a predetermined steering angle using the ultrasound probe and the non-steered transmission/reception image that is the image of the breast via the compression plate by non-beam steering using the ultrasound probe are created in a state where the breast is compressed by the compression plate, and the compound image is created by using the created steered transmission/reception image and the created non-steered transmission/reception image.

Here, the console 50 according to the present embodiment applies an image created by using, as a main frequency component, a frequency component in a range in which an amplitude of an artifact caused by a sidelobe of the ultrasound beam is half or less of an amplitude of the ultrasound beam transmitted to create the steered transmission/reception image, as the steered transmission/reception image, and applies an image created by using the frequency component as the main frequency component, as the non-steered transmission/reception image. Therefore, it is possible to effectively suppress an artifact caused by multiple reflections of an ultrasound beam in a compression plate in a case of performing ultrasound imaging of a breast through the compression plate, as compared with the related art.

In addition, with the console 50 according to the present embodiment, the compound image is created in a harmonic mode or a compound harmonic mode. Therefore, in a case where the compound image is created in the harmonic mode or the compound harmonic mode, the artifact caused by the multiple reflections can be suppressed.

In addition, with the console 50 according to the present embodiment, in a case where the compound image is created in the compound harmonic mode, the weight of the harmonic component for a shallow part including a portion of the multiple reflections of the ultrasound beam is increased as compared with the weight of the fundamental wave component, and the weight of the fundamental wave component is increased as the depth increases from the shallow part to a deep part. Therefore, a high-definition image can be obtained in the shallow part, and the penetration in the deep part can be secured, so that the compound image can be improved in quality.

In addition, with the console 50 according to the present embodiment, the setting to create the compound image is changed in a case where the compression by the compression plate is ended. Therefore, unnecessary ultrasound imaging in a state where the compression by the compression plate is not ended can be avoided.

In addition, with the console 50 according to the present embodiment, the compound image is created by changing the ratio of the weights of the steered transmission/reception image and the non-steered transmission/reception image according to the depth of the multiple reflections of the ultrasound beam. Therefore, it is possible to reduce the influence of the multiple reflections or to improve the image quality (resolution, reduction of artifacts other than the multiple reflections, and the like) of the entire compound image.

In addition, with the console 50 according to the present embodiment, the compound image is created by not using a reflected wave corresponding to between a front surface that is a surface of the compression plate on a side opposite to the breast and a back surface that is a surface of the compression plate on the side of the breast, increasing a weight of the steered transmission/reception image as compared with a weight of the non-steered transmission/reception image from the back surface of the compression plate to a depth of the multiple reflections of the ultrasound beam, and increasing the weight of the non-steered transmission/reception image as the depth increases. Therefore, it is possible to more effectively reduce the influence of the multiple reflections or to improve the image quality of the entire compound image.

Further, with the console 50 according to the present embodiment, the sound speed of the ultrasound beam is set to the sound speed between the sound speed of the breast and the sound speed of the compression plate. Therefore, it is possible to improve the resolution of the compound image.

In the above-described embodiment, a case where the CPU 51 provided in the console 50 is applied as the processor according to the technique of the present disclosure has been described, but the present disclosure is not limited to this. For example, a form may be adopted in which a CPU of the control unit 20 provided in the image generation apparatus 10 is applied as the processor according to the technology of the present disclosure.

In addition, in the above-described embodiment, a case has been described in which the setting is changed to create the compound image in a case where the compression by the compression member 40 is ended, but the present disclosure is not limited to this. For example, a form in which the setting is changed to create the compound image in a case where the imaging mode using the compression member 40 is selected may be adopted. In addition, for example, a form in which the setting is changed to create the compound image in a case where an image corresponding to the compression member 40 is recognized in at least one of the steered transmission/reception image or the non-steered transmission/reception image may be adopted.

That is, in the image generation apparatus 10, the radiation image and the ultrasound image can be captured, but in some cases, a mode in which the compression member 40 is used and a mode in which the compression member 40 is not used are provided in advance as the modes for capturing the ultrasound image. In this case, in a case where the ultrasound imaging is performed in the latter imaging mode, the multiple reflections caused by the compression member 40 do not occur, so that it is not necessary to create the compound image according to the embodiment. Therefore, in this case, by creating the compound image according to the embodiment only in a case where the imaging mode using the compression member 40 is selected, unnecessary processing can be avoided.

In addition, as a method of recognizing the image corresponding to the compression member 40 in at least one of the steered transmission/reception image or the non-steered transmission/reception image, a method using pattern matching, a machine-learned image extraction model in advance, or the like can be applied.

In the above-described embodiment, for example, as a hardware structure of a processing unit that executes various types of processing such as the first creation unit 60, the second creation unit 62, and the display control unit 64, various processors shown below can be used. The various processors include, as described above, in addition to a CPU, which is a general-purpose processor that functions as various processing units by executing software (program), a programmable logic device (PLD) that is a processor of which a circuit configuration may be changed after manufacture, such as a field programmable gate array (FPGA), and a dedicated electrical circuit which is a processor having a circuit configuration specially designed to execute specific processing, such as an application specific integrated circuit (ASIC).

One processing unit may be configured of one of the various processors, or may be configured of a combination of the same or different kinds of two or more processors (for example, a combination of a plurality of FPGAs or a combination of the CPU and the FPGA). Further, a plurality of processing units may be configured by one processor.

As an example in which a plurality of processing units are configured of one processor, first, as typified by a computer such as a client or a server, there is an aspect in which one processor is configured of a combination of one or more CPUs and software, and this processor functions as a plurality of processing units. A second example is a form of using a processor that implements the function of the entire system including the plurality of processing units via one integrated circuit (IC) chip, as represented by a system on a chip (SoC) or the like. As described above, the various processing units are configured by using one or more of the above various processors as the hardware structure.

Further, as the hardware structure of the various processors, more specifically, an electric circuit (circuitry) in which circuit elements such as semiconductor elements are combined may be used.

In addition, in the above-described embodiment, the aspect is described in which the image processing program 52A is stored (installed) in the storage unit 52 of the console 50 in advance, but the present disclosure is not limited thereto. The image processing program 52A may be provided in a form recorded in a recording medium, such as a compact disc read-only memory (CD-ROM), a digital versatile disc read-only memory (DVD-ROM), and a universal serial bus (USB) memory. In addition, the image processing program 52A may be downloaded from an external device via a network.

In addition, the present disclosure can also be applied to a program and a program product. Specifically, the image processing program 52A in each of the above-described embodiments may be provided as a program product. The program product includes all forms of products for providing a program. For example, the program product includes a program provided through a network such as the Internet, and non-transitory computer-readable recording media such as a CD-ROM and a DVD in which the program is stored.

From the above description, the invention according to the following supplementary notes can be understood.

### Supplementary Note 1

An image processing apparatus comprising:
a processor,
wherein the processor is configured to:
   create a steered transmission/reception image that is an image of a breast obtained by an ultrasound beam which is transmitted and received, via a compression plate, by beam steering with a predetermined steering angle using an ultrasound probe and a non-steered transmission/reception image that is an image of the breast obtained by the ultrasound beam which is transmitted and received, via the compression plate, by non-beam steering using the ultrasound probe, in a state where the breast is compressed by the compression plate; and
   create a compound image using the created steered transmission/reception image and the created non-steered transmission/reception image,
the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and
the non-steered transmission/reception image is an image created by using the frequency component as the main frequency component.

### Supplementary Note 2

The image processing apparatus according to Supplementary Note 1,
wherein the processor is configured to create the compound image in a harmonic mode or a compound harmonic mode.

### Supplementary Note 3

The image processing apparatus according to Supplementary Note 2,
wherein in a case where the compound image is created in the compound harmonic mode, the processor is configured to:
   increase a weight of a harmonic component for a shallow part including a portion of multiple reflections of the ultrasound beam, as compared with a weight of a fundamental wave component; and
   increase the weight of the fundamental wave component as a depth increases from the shallow part to a deep part.

### Supplementary Note 4

The image processing apparatus according to any one of Supplementary Notes 1 to 3,
wherein the processor is configured to change a setting to create the compound image in a case where an imaging mode using the compression plate is selected.

### Supplementary Note 5

The image processing apparatus according to any one of Supplementary Notes 1 to 3,
wherein the processor is configured to change a setting to create the compound image in a case where an image corresponding to the compression plate is recognized in at least one of the steered transmission/reception image or the non-steered transmission/reception image.

### Supplementary Note 6

The image processing apparatus according to any one of Supplementary Notes 1 to 3,
wherein the processor is configured to change a setting to create the compound image in a case where the compression by the compression plate is ended.

### Supplementary Note 7

The image processing apparatus according to any one of Supplementary notes 1 to 6,
wherein the processor is configured to create the compound image by changing a ratio of weights of the steered transmission/reception image and the non-steered transmission/reception image depending on a depth of multiple reflections of the ultrasound beam.

### Supplementary Note 8

The image processing apparatus according to any one of Supplementary Notes 1 to 6,
wherein the processor is configured to create the compound image by not using a reflected wave corresponding to between a front surface that is a surface of the compression plate on a side opposite to the breast and a back surface that is a surface of the compression plate on the side of the breast, increasing a weight of the steered transmission/reception image as compared with a weight of the non-steered transmission/reception image from the back surface of the compression plate to a depth of multiple reflections of the ultrasound beam, and increasing the weight of the non-steered transmission/reception image as the depth increases.

### Supplementary Note 9

The image processing apparatus according to any one of Supplementary Notes 1 to 8,
wherein a sound speed of the ultrasound beam is a sound speed between a sound speed of the breast and a sound speed of the compression plate.

### Supplementary Note 10

The image processing apparatus according to any one of Supplementary Notes 1 to 9,
wherein the main frequency component is a frequency component of 8 MHz or less, and
the steering angle is an angle of 20 degrees or less.

### Supplementary Note 11

The image processing apparatus according to any one of Supplementary Notes 1 to 9,
wherein the main frequency component is a frequency component of 9 MHz or less, and
the steering angle is an angle of 15 degrees or less.

### Supplementary Note 12

The image processing apparatus according to any one of Supplementary Notes 1 to 9,
wherein the main frequency component is a frequency component of 11 MHz or less, and
the steering angle is an angle of 10 degrees or less.

### Supplementary Note 13

A medical image acquisition apparatus comprising:
the image processing apparatus according to any one of Supplementary Notes 1 to 12; and
a display unit that displays the compound image created by the image processing apparatus.

### Supplementary Note 14

A program causing a computer to execute a process comprising:
creating a steered transmission/reception image that is an image of a breast obtained by an ultrasound beam which is transmitted and received, via a compression plate, by beam steering with a predetermined steering angle using an ultrasound probe and a non-steered transmission/reception image that is an image of the breast obtained by the ultrasound beam which is transmitted and received, via the compression plate, by non-beam steering using the ultrasound probe, in a state where the breast is compressed by the compression plate; and
creating a compound image using the created steered transmission/reception image and the created non-steered transmission/reception image,
in which the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and
the non-steered transmission/reception image is an image created by using the frequency component as the main frequency component.

The disclosure of JP2023-168674 filed on Sep. 28, 2023 is incorporated herein by reference in its entirety. All documents, patent applications, and technical standards mentioned in the present specification are incorporated herein by reference to the same extent as in a case where each document, each patent application, and each technical standard are specifically and individually described by being incorporated by reference.

What is claimed is:

1. An image processing apparatus comprising a processor, wherein:
the processor is configured to:
create a steered transmission/reception image that is an image of a breast obtained by an ultrasound beam which is transmitted and received, via a compression plate, by beam steering with a predetermined steering angle using an ultrasound probe and a non-steered transmission/reception image that is an image of the breast obtained by the ultrasound beam which is transmitted and received, via the compression plate, by non-beam steering using the ultrasound probe, in a state where the breast is compressed by the compression plate; and
create a compound image using the created steered transmission/reception image and the created non-steered transmission/reception image,
the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and
the non-steered transmission/reception image is an image created by using the frequency component as the main frequency component.

2. The image processing apparatus according to claim 1, wherein the processor is configured to create the compound image in a harmonic mode or a compound harmonic mode.

3. The image processing apparatus according to claim 2, wherein in a case where the compound image is created in the compound harmonic mode, the processor is configured to:
increase a weight of a harmonic component for a shallow part including a portion of multiple reflections of the ultrasound beam, as compared with a weight of a fundamental wave component; and
increase the weight of the fundamental wave component as a depth increases from the shallow part to a deep part.

4. The image processing apparatus according to claim 1, wherein the processor is configured to change a setting to create the compound image in a case where an imaging mode using the compression plate is selected.

5. The image processing apparatus according to claim 1, wherein the processor is configured to change a setting to create the compound image in a case where an image corresponding to the compression plate is recognized in at least one of the steered transmission/reception image or the non-steered transmission/reception image.

6. The image processing apparatus according to claim 1, wherein the processor is configured to change a setting to create the compound image in a case where the compression by the compression plate is ended.

7. The image processing apparatus according to claim 1, wherein the processor is configured to create the compound image by changing a ratio of weights of the steered transmission/reception image and the non-steered transmission/reception image depending on a depth of multiple reflections of the ultrasound beam.

8. The image processing apparatus according to claim 1, wherein the processor is configured to create the compound image by not using a reflected wave corresponding to between a front surface that is a surface of the compression plate on a side opposite to the breast and a back surface that is a surface of the compression plate on the side of the breast, increasing a weight of the steered transmission/reception image as compared with a weight of the non-steered transmission/reception image from the back surface of the compression plate to a depth of multiple reflections of the ultrasound beam, and increasing the weight of the non-steered transmission/reception image as the depth increases.

9. The image processing apparatus according to claim 1, wherein a sound speed of the ultrasound beam is a sound speed between a sound speed of the breast and a sound speed of the compression plate.

10. The image processing apparatus according to claim 1, wherein:
the main frequency component is a frequency component of 8 MHz or less, and
the steering angle is an angle of 20 degrees or less.

11. The image processing apparatus according to claim 1, wherein:
the main frequency component is a frequency component of 9 MHz or less, and
the steering angle is an angle of 15 degrees or less.

12. The image processing apparatus according to claim 1, wherein:
the main frequency component is a frequency component of 11 MHz or less, and
the steering angle is an angle of 10 degrees or less.

13. A medical image acquisition apparatus comprising:
the image processing apparatus according to claim 1; and
a display unit that displays the compound image created by the image processing apparatus.

14. A non-transitory computer-readable storage medium storing a program causing a computer to execute a process comprising:
creating a steered transmission/reception image that is an image of a breast obtained by an ultrasound beam which is transmitted and received, via a compression plate, by beam steering with a predetermined steering angle using an ultrasound probe and a non-steered transmission/reception image that is an image of the breast obtained by the ultrasound beam which is transmitted and received, via the compression plate, by non-beam steering using the ultrasound probe, in a state where the breast is compressed by the compression plate; and
creating a compound image using the created steered transmission/reception image and the created non-steered transmission/reception image,
in which the steered transmission/reception image is an image created by using, as a main frequency component, a frequency component in a range in which an amplitude of an artifact caused by a sidelobe of the ultrasound beam is half or less of an amplitude of the ultrasound beam transmitted to create the steered transmission/reception image, and
the non-steered transmission/reception image is an image created by using the frequency component as the main frequency component.
